(19) United States
(12) Patent Application Publication
(10) Pub. No.: US 2015/0297143 A1
(43) Pub. Date: Oct. 22, 2015

(54) ASSESSING PATIENT RISK FOR AN ACUTE HYPOTENSIVE EPISODE

(71) Applicant: XEROX CORPORATION, Norwalk, CT (US)

(72) Inventors: Vaibhav RAJAN, Bangalore (IN); Sakyajit BHATTACHARYA, Bangalore (IN); Vijay HUDDAR, Bijapur (IN)

(73) Assignee: XEROX CORPORATION, Norwalk, CT (US)

(21) Appl. No.: 14/254,232

(22) Filed: Apr. 16, 2014

(51) Int. Cl. A61B 5/00; G06N 99/00; G06F 19/00; A61B 5/021

(52) U.S. Cl. CPC A61B 5/7267; A61B 5/021; G06N 99/005; G06F 19/322

(57) ABSTRACT

What is disclosed is a system and method for assessing patient risk for an acute hypotensive episode. In one embodiment, the present method involves retrieving a training set from a database. The training set comprises mean arterial pressures (MAPs) for a plurality of subjects. Each MAP comprises systolic and diastolic measurements. The training set is used to train the present classifier system. Once trained, the present classifier system classifies an unclassified patient into either a first class or a second class. The first class is at risk for an acute hypotensive episode occurring within a prediction window of w≧60 minutes in the future. The second class is not at risk for an acute hypotensive episode. A MAP of an unclassified patient is retrieved or otherwise obtained. Thereafter, the present classifier system proceeds to classify the patient into the first or second class. Various embodiments are disclosed.

### TECHNICAL FIELD

The present invention is directed to systems and methods for assessing a patient's risk for the sudden onset of a period of sustained low blood pressure, referred to as an acute hypotensive episode.

### BACKGROUND

An acute hypotensive episode (AHE) is generally defined as the sudden onset of a period of sustained low blood pressure. Acute hypotensive episodes are an important precursor to various health complications, some of which may be life threatening. It is one of the most critical conditions in an intensive care unit (ICU) for patient monitoring. Left untreated, AHE can lead to organ damage and may even cause death. If AHE can be predicted in advance then timely intervention can prevent such complications and may save the patient's life. Much work has been done trying to predict AHE. Automated systems have arisen to prospectively identify patients who are at risk for an acute hypotensive episode. It is highly desirable in this art to predict an occurrence of an acute hypotensive episode to improve intervention and increase patient survival, particularly in ICUs. The teachings hereof are directed towards this effort.

Accordingly, what is needed in this art are sophisticated systems and methods for assessing patient risk for an occurrence of an acute hypotensive episode within the timeframe of a prediction window in the future.

### BRIEF SUMMARY

What is disclosed is a system and method for assessing patient risk for an occurrence of an acute hypotensive episode within the timeframe of a prediction window in the future. In one embodiment, the present method involves the following. A training set is retrieved from a database. The training set comprises mean arterial pressures (MAPs) for a plurality of subjects. Each MAP is calculated from systolic and diastolic measurements. The training set is used to train the present classifier system. The present classifier system, as disclosed herein further in detail, functions to classify a yet unclassified patient into either a first or a second class. In the first class, the patient is identified as being at risk for sudden onset of a period of sustained low blood pressure (an acute hypotensive episode) within the timeframe of a prediction window of w minutes in the future. In the second class, the patient is identified as not being at risk for the occurrence of an acute hypotensive episode. Once the present system has been trained, a MAP of a previously unclassified patient is retrieved or otherwise obtained. The present classifier system is then used to classify that patient into one of the first and second classes. Various embodiments are disclosed.

Features and advantages of the above-described method will become readily apparent from the following detailed description and accompanying drawings.

### BRIEF DESCRIPTION OF THE DRAWINGS

The foregoing and other features and advantages of the subject matter disclosed herein will be made apparent from the following detailed description taken in conjunction with the accompanying drawings, in which:

FIG. 1 is a flow diagram which illustrates one embodiment of the present method for assessing patient risk for an acute hypotensive episode in accordance with the teachings hereof;

FIG. 2 is a continuation of the flow diagram of FIG. 1 with flow processing continuing with respect to node A; and

FIG. 3 is a block diagram of one example system for performing various aspects of the present method as described with respect to the flow diagrams of FIGS. 1-2.

### DETAILED DESCRIPTION

What is disclosed is a system and method for assessing patient risk for an occurrence of an acute hypotensive episode within the timeframe of a prediction window in the future.

### NON-LIMITING DEFINITIONS

A “subject” refers to refers to a person being monitored for an acute hypotensive episode. The terms “subject” and “patient” are used interchangeably. The subject is typically a patient in an intensive care unit (ICU).

An “unclassified patient” is a person who is being classified by the present classifier system.

“Arterial blood pressure” or simply “blood pressure”, is a vital sign that is routinely monitored. Blood pressure is written as a ratio in mmHg (e.g. 120/80). The top number (systolic), which is also the higher of the two numbers, is a measure of the pressure in the arteries when the heart beats (i.e., when the heart muscle contracts). The bottom number (diastolic), which is also the lower of the two numbers, is a measure of the pressure in the arteries between heartbeats (i.e., when the heart muscle is resting between beats and refilling with blood). Systolic and diastolic blood pressures are typically measured using a device called a sphygmomanometer. Normal blood pressure is in the range of 90/60 to 130/80 mmHg. Hypertension (high blood pressure) is often defined when the subject's blood pressure rises above the normal range, i.e., greater than 130/80 mmHg. Conversely, hypotension (low blood pressure) is often defined when the subject's blood pressure falls below the normal range, i.e., less than 90/60 mmHg. Both hypertension and hypotension are important mortality predictors for patients with cardiovascular abnormalities and are precursors often leading to patient death in the ICU.

An “acute hypotensive episode” (AHE) also called an “acute hypotensive event”, is defined herein as a period of 30 minutes or longer during which at least 90% of the non-overlapping one minute averages of the arterial blood pressure waveform are under 60 mmHg. A variety of different conditions can cause AHE including sepsis, myocardial infarction, cardiac arrhythmia, pulmonary embolism, hemorrhage, dehydration, anaphylaxis, hypovolemia, insufficient cardiac output, vasodilatory shock, and the effects of medication. In many cases, an acute hypotensive episode is a precursor to other complications. If an acute hypotensive episode can be predicted in advance then timely intervention can prevent complications and death. The present method utilizes mean arterial pressure which is monitored in ICUs.

“Mean Arterial Pressure” (MAP) is a function of systolic and diastolic pressure and is considered to be the perfusion pressure seen by organs in the body. MAP is be approximated from systolic and diastolic pressure measurements as follows:

$$M A P ≅ \frac{2}{3} DP - \frac{1}{3} SP \qquad ( 1 )$$

where DP is the diastolic pressure and SP is the systolic pressure. Diastolic pressure counts twice as much as systolic because 23 of the cardiac cycle is spent in diastole when the heart muscle is resting between beats and refilling with blood. Mean arterial pressure is approximated. To determine mean arterial pressure with absolute accuracy, electronic equipment needs to be employed.

“Obtaining a MAP” is intended to be widely construed and includes: retrieving, receiving, capturing, calculating or otherwise acquiring one or more MAPs for processing in accordance with the classifier system disclosed herein. MAPs can be obtained from a memory, storage device, or from a media such as a CDROM, DVD, and the like. MAPs can be calculated by hand and entered manually into a record or data format used by the present classifier system. MAPs can be obtained from a remote device over a network or downloaded from a web-based system or application which makes MAPs available to the present classifier system.

A “training set”, as used herein, refers to mean arterial blood pressures of a plurality of subjects. The training set can be obtained from a database such as, for instance, MIMIC-II (Multi-parameter Intelligent Monitoring in Intensive Care) database. The MIMIC-II database comprises well-characterized physiologic signals and clinical data of more than 2300 ICU patients. MIMIC-II is a publicly available archive for use by the biomedical research community as part of PhysioNet. Vital signs for most of the ICU patients in the training set have been sampled on time intervals of every minute. Each record consists of a time series signal xₜ, t=1, . . . , N, where N is the total number of MAP measurements for the patient, xₜ is the MAP measurement at time t, time being reckoned from the start of the measurements in the ICU at an interval of 1 minute. In one embodiment, an acute hypotensive episode is defined as an interval at time i in the time-series signal xₜ where [xᵢ; xᵢ+30] wherein at least 27 values comprising the patient's MAP are not greater than 60. Not all the records are usable since many of them have missing or erroneous data. Records were discarded that contain less than 6 hours of MAP measurements including those that contained acute hypotensive episodes in the first 5 hours of recorded data. From the remaining records, we selected 1700 records of ICU patients out of which 600 contained an acute hypotensive episode (first class) and 1100 did not contain an acute hypotensive episode (second class). The training set used herein comprised 500 records of subjects from the first class and 1000 records of subjects from the second class. Other records were put aside and used for development and testing purposes. It should be appreciated that, as new data points of become available, that data is added to the training set. The training set is used in accordance with the teachings hereof to train the classifier system disclosed herein.

A “prediction window” is a period of time in the future such as, for instance w≧60 minutes, when an acute hypotensive episode is likely to occur.

The “classifier system” disclosed herein, in one embodiment, comprises at least a processor and a memory. The processor retrieves machine readable program instructions from memory and executes those instructions causing the processor to classify an unclassified patient into a first or a second class. In the first class, the patient is identified as being at risk for an acute hypotensive episode occurring within the timeframe of the prediction window. In the second class, the patient is identified as not being at risk for an acute hypotensive episode.

The present classifier system performs in accordance with the following rules.
a) Let a first vector y₁ consist of MAPs of subjects in the first class.
b) Let a second vector y₂ consist of MAPs of subjects in the second class.
c) Let μ₁=mean(y₁) and μ₂=mean(y₂).
d) Let σ₁=sd(y₁) and σ₂=sd (y₂).
e) Let k be a value such that μ₁+kσ₁<μ₂−kσ₂.
f) Let n₁ be the number of y₁ values above (μ₂−kσ₂).
g) Let n₂ be the number of y₁ values within a range of (μ₁+kσ₁, μ₂−kσ₂).
h) Let n₃ be the number of y₂ values below (μ₁+kσ₁).
i) Let n₄ be the number of y₂ values within a range of (μ₁+kσ₁, μ₂−kσ₂).
j) Let k₀=min(n₁+n₂+n₃+n₄).

If the mean of the unclassified patient's MAPs averaged over at least a one hour time interval immediately preceding the start of the prediction window is less than (μ₁+k₀σ₁), then the present classifier system classifies the patient into the first class, i.e., this patient is at risk for the occurrence of an acute hypotensive episode happening within the timeframe of a prediction window of w minutes in the future.

If the mean of the unclassified patient's MAPs averaged over at least a one hour time interval immediately preceding the start of the prediction window is greater than (μ₂−k₀σ₂), then the present classifier system classifies this patient into the second class, i.e., this patient is not at risk for an acute hypotensive episode.

If the mean of the unclassified patient's MAPs averaged over at least a one hour time interval immediately preceding the start of the prediction window falls within the range of (μ₁+kσ₁, μ₂−kσ₂), then the mean squared deviations of a last of the MAP measurements from all points in the first and second vectors y₁, y₂ are calculated, given as d₁, d₂, respectively. If d₁>d₂ then the present classifier system classifies this patient into the second class. Otherwise, this patient is classified into the first class.

Determining k₀.

Let Y₁, Y₂, Y₃, Y₄ denote vectors of indicator variables (which take values 0 or 1) with lengths |Y₁|=|Y₂|=|y₁| and |Y₃|=|Y₄|=|y₂|. After the following computation, Y₁ will contain n₁ 1's indicating those values in y₁ that are greater than μ₂−kσ₂, and Y₂ with contain n₂ 1's indicating those values in y₁ that lie in the range (μ₁+kσ₁, μ₂−kσ₂). Similarly, Y₃ and Y₄ contain indicator variables with respect to values in y₂. If we denote the j^{th} element of vector Y₁ for iε{1,2,3,4} by x_{i,j} and the j^{th} element of vector y₁ for iε{1,2} by y_{i,j} then k₀ can be obtained by minimizing the following relationship:

$${\sum }_{i = 1}^{\vert {y}_{1} \vert} {x}_{1 i} + {\sum }_{i = 1}^{\vert {y}_{1} \vert} {x}_{2 i} + {\sum }_{j = 1}^{\vert {y}_{2} \vert} {x}_{3 j} + {\sum }_{j = 1}^{\vert {y}_{2} \vert} {x}_{4 j} \qquad ( 2 )$$

subject to these constraints:

X₁ᵢy₁ᵢ+kσ₂>μ₂ (3)

x₂ᵢY₁ᵢ−kσ₁>μ₁ (4)

x₂ᵢy₁ᵢ+kσ₂<μ₂ (5)

x₃ⱼy₂ⱼ−kσ₁<μ₁ (6)

x₄ⱼy₂ⱼ−kσ₁>μ₁ (7)

x₄ⱼy₂ⱼ−kσ₂<μ₂ (8)

x₁ᵢε{0,1},x₂ᵢε{_{0,1}} (9)

x₃ⱼε{0,1},x₄ⱼε{0,1} (10)

for 1≦i≦|y₁|, 1≦j≦|y₂|, k≧0, and k(σ₁+σ₂)<μ₂−μ₁.

Standard optimization suites commonly found in the arts, such as CPLEX, can be utilized to solve the above-described minimization of Eq. (2). To minimize both false positives and false negatives, where a true positive is a correct identification in the first class, an interval-based margin is built which is based on the mean and standard deviation values in the first and second classes, as seen in the training data: (μ₁+kσ₁, μ₂−kσ₂). A value falling below the lower boundary of the interval is considered to be in the first class and a value above the upper boundary is considered to be in the second class. By choosing the right value of k, the sum of the number of first class values falling above the lower bound of (n₁+n₂) and the number of second class values falling below the upper bound of (n₃+n₄) is effectively minimized.

It should be appreciated that the present classifier system can be run in a batch mode or in an incremental mode. As new training values arrives in both classes, vectors y₁ and y₂ are updated as well as the mean (μ₁,μ₂) and standard deviations (σ₁,σ₂). If we let x be the sample added in the n^{th} round, μₜ and μₜ² denote the mean and variance, respectively, in the t^{th} round, the following can be used to compute the mean and variance in a real-time manner.

$$\begin{matrix}{μ}_{n} = {μ}_{n - 1} + \frac{x - {μ}_{n - 1}}{n} & ( 11 ) \\ {σ}_{n}^{2} = \frac{( n - 1 ) {σ}_{n - 1}^{2} + ( x - {μ}_{n - 1} ) ( x - {μ}_{n} )}{n} & ( 12 )\end{matrix}$$

### Flow Diagram of One Embodiment

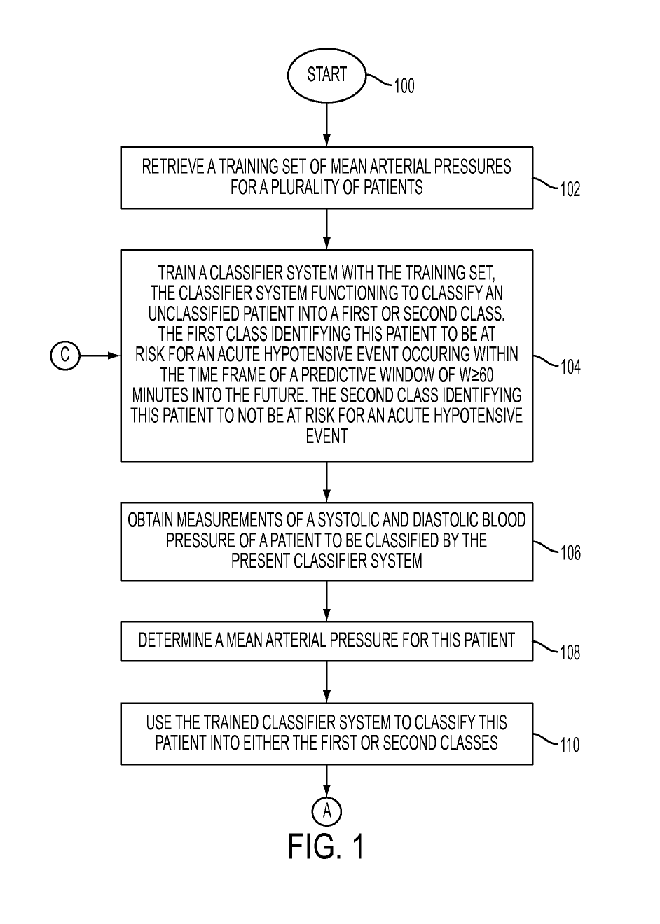

Reference is now being made to the flow diagram of FIG. 1 which illustrates one embodiment of the present method for assessing patient risk for an acute hypotensive episode in accordance with the teachings hereof. Flow processing begins at step 100 and immediately proceeds to step 102.

At step 102, retrieve a training set of mean arterial pressures for a plurality of subjects (preferably while these patients were in intensive care units). Each mean arterial pressure is estimated from systolic and diastolic pressures measured at a time t.

At step 104, train a classifier system with the training set which functions to classify an unclassified patient into a first class identifying this patient to be at risk for sudden onset of an acute hypotensive episode within the timeframe of a prediction window of w≧60 minutes in the future, and a second class identifying this patient to not be at risk for the occurrence of an acute hypotensive episode.

At step 106, obtain measurements of a systolic and diastolic blood pressure of a patient to be classified by the present classifier system. Systolic and diastolic blood pressure measurements can be readily obtained using a sphygmomanometer, as is commonly understood in the medical device arts.

At step 108, determine a mean arterial pressure for this patient. One embodiment for calculating mean arterial pressure is given in Eq. (1).

At step 110, use the trained classifier system to classify this patient into either the first or second classes.

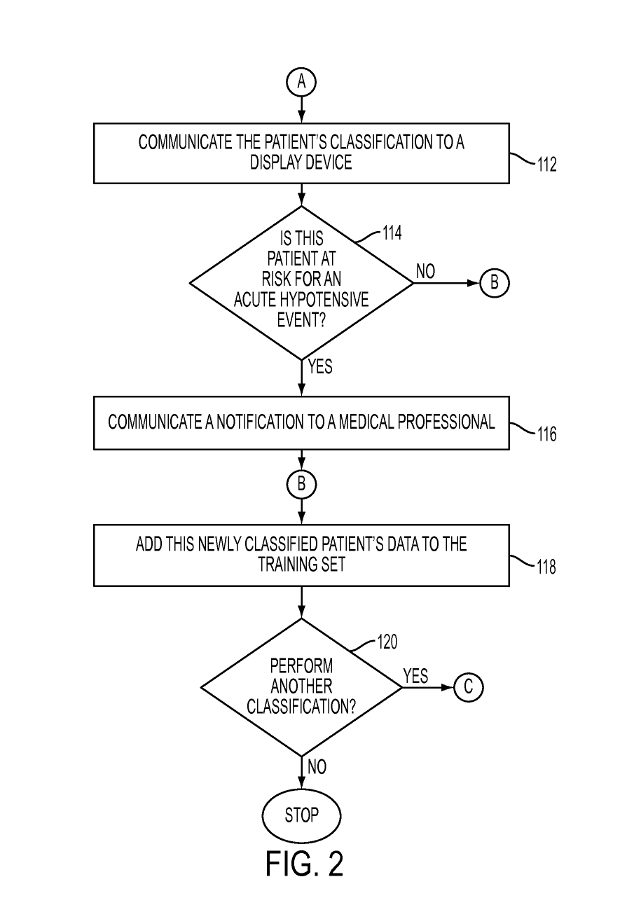

Reference is now being made to the flow diagram of FIG. 2 which is a continuation of the flow diagram of FIG. 1 with flow processing continuing with respect to node A.

At step 112, communicate the patient's classification to a display device. In other embodiments, the patient classification is communicated to any of: a storage device, a wireless handheld device, a laptop, tablet-PC, and a workstation.

At step 114, a determination is made whether the classified patient is at risk for an acute hypotensive episode occurring within the timeframe of a prediction window. If so then, at step 116, communicate a notification to a medical profession. In various embodiments hereof, the notification can take the form of an audio message, a text message, an email, a phone call, or a video. The notification may be an alert signal which takes the form of a message displayed on a display device or a sound activated at, for example, a nurse's station or control panel. The alert may take the form of a colored or blinking light which provides a visible indication that an alert condition exists. The alert signal may be communicated to one or more remote devices over a wired or wireless network. The alert may be sent directly to a handheld wireless cellular device of a medical professional. Thereafter, additional actions would be taken in response to the alert signal. Otherwise, if the patient is not at risk for an acute hypotensive episode occurring within the timeframe of a prediction window then processing continues with respect to node B, at step 118, add this newly classified patient's data to the training set.

At step 120, a determination is made whether to perform another classification. If so then processing repeats with respect to node C wherein, at step 205, the training set is used to once again train the present classifier system. Processing repeats in a similar manner. If it is determined that further patient classification is not to be performed then, in this embodiment, further processing stops.

It should also be appreciated that the flow diagrams depicted herein are illustrative. One or more of the operations illustrated in the flow diagrams may be performed in a differing order. Other operations may be added, modified, enhanced, or consolidated. Variations thereof are intended to fall within the scope of the appended claims.

### Block Diagram of System

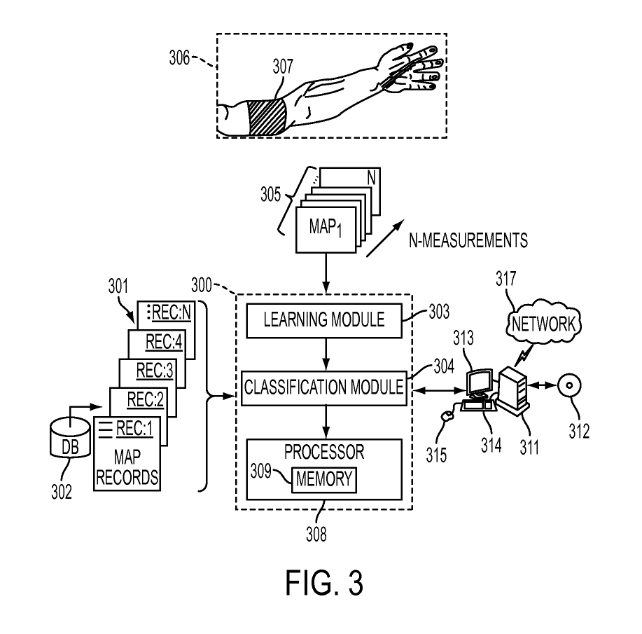

Reference is now being made to FIG. 3 which shows a block diagram of one example system for performing various aspects of the present method as described with respect to the flow diagrams of FIGS. 1-2.

In FIG. 3, a training set (collectively at 301) comprising records containing mean arterial pressures (MAPs) for a plurality of subjects are retrieved from a database 302. Database 302 is a storage device wherein records are stored, manipulated, and retrieved in response to a query. Such records, in various embodiments, take the form of patient medical histories stored in association with information identifying the patient along with medical information. Although the database is shown as an external device, the database may be internal to the workstation 311 mounted, for example, on a hard disk therein. The training set is provided to the classifier system 300 for training purposes. The MAPs of the training set are obtained from systolic and diastolic measurements taken in a range of 10-60 minutes immediately preceding a start of a prediction window for both first and second classes.

In the embodiment shown, the classifier system 300 comprises a plurality of modules. Learning Module 303 processes the training data contained in the records of the training set such that the classifier system can be trained. The Learning Module 303 further functions to prune the training set, as desired, such that the classifier is trained with data which meet a pre-determined criteria, at least for accuracy and timeliness. Once training has completed, Learning Module 303 signals Classification Module 304 to receive a total of n MAP measurements (collectively at 305) where n≧1 of the yet-to-be classified patient shown as a snapshot 306 of the unclassified patient's right arm. The blood pressure measurements comprising the patient's MAP are obtained using a pressure cuff 307, as are generally understood, of a sphygmomanometer device (not shown). The unclassified patient's MAPs are received or are otherwise obtained by the classifier system 300 which, in turn, proceeds to classify the unclassified patient into one of: a first class where the patient is identified as being at risk for the occurrence of acute hypotensive episode (AHE) within the timeframe of a prediction window in the future, and a second class where the patient is identified as not being at risk for an acute hypotensive episode. Processor 308 retrieves machine readable program instructions from Memory 309 and is provided to facilitate the functionality of the various modules comprising the classifier system 300. The processor, operating alone or in conjunction with other processors and memory, may be configured to assist or otherwise facilitate the functionality of any of the processors and modules of system 300.

The classifier system of FIG. 3 is shown in communication with a workstation 311. A computer case of the workstation houses various components such as a motherboard with a processor and memory, a network card, a video card, a hard drive capable of reading/writing to machine readable media 312 such as a floppy disk, optical disk, CD-ROM, DVD, magnetic tape, and the like, and other software and hardware needed to perform the functionality of a computer workstation. The workstation further includes a display device 313, such as a CRT, LCD, or touchscreen device, for displaying information, video, measurement data, computed values, medical information, results, locations, and the like. A user can view any of that information and make a selection from menu options displayed thereon. Keyboard 314 and mouse 315 effectuate a user input.

It should be appreciated that the workstation 311 has an operating system and other specialized software configured to display alphanumeric values, menus, scroll bars, dials, slideable bars, pull-down options, selectable buttons, and the like, for entering, selecting, modifying, and accepting information needed for processing in accordance with the teachings hereof. The workstation is further enabled to display MAPs and patient classifications as they are derived. The workstation may further display interim values, boundary conditions, and the like, in real-time as the classifier system 300 performs its intended functionality as described herein in detail.

A user or technician may use the user interface of the workstation to set parameters, view/adjust/delete values in the training set, and adjust various aspects of the classifier system 300 as needed or as desired, depending on the implementation. Any of these selections or input may be stored/retrieved to storage device 312. Default settings can be retrieved from the storage device. A user of the workstation is also able to view or manipulate any of the records contained in the training set 301 via pathways not shown.

Although shown as a desktop computer, it should be appreciated that the workstation 311 can be a laptop, mainframe, or a special purpose computer such as an ASIC, circuit, or the like. The embodiment of the workstation of FIG. 3 is illustrative and may include other functionality known in the arts. Any of the components of the workstation may be placed in communication with the classifier system 300 or any devices in communication therewith. Any of the modules of the classifier system 300 can be placed in communication with storage device 302 and/or computer readable media 312 and may store/retrieve therefrom data, variables, records, parameters, functions, and/or machine readable/executable program instructions, as needed to perform their intended functions. Each of the modules of the classifier system 300 may be placed in communication with one or more remote devices over network 317.

It should be appreciated that some or all of the functionality performed by any of the modules or processing units of the video processing system can be performed, in whole or in part, by the workstation 311 placed in communication with the classifier system 300 over network 317. The embodiment shown is illustrative and should not be viewed as limiting the scope of the appended claims strictly to that configuration. Various modules may designate one or more components which may, in turn, comprise software and/or hardware designed to perform the intended function.

The teachings hereof can be implemented in hardware or software using any known or later developed systems, structures, devices, and/or software by those skilled in the applicable art without undue experimentation from the functional description provided herein with a general knowledge of the relevant arts. One or more aspects of the methods described herein are intended to be incorporated in an article of manufacture which may be shipped, sold, leased, or otherwise provided separately either alone or as part of a product suite or a service.

It will be appreciated that the above-disclosed and other features and functions, or alternatives thereof, may be desirably combined into other different systems or applications. Presently unforeseen or unanticipated alternatives, modifications, variations, or improvements may become apparent and/or subsequently made by those skilled in this art which are also intended to be encompassed by the following claims. The teachings of any publications referenced herein are each hereby incorporated by reference in their entirety.

What is claimed is:

1. A method for assessing patient risk for an acute hypotensive episode, the method comprising:
using a training set of mean arterial pressures (MAPs) for a plurality of subjects to train a classifier system, said classifier system classifying an unclassified patient into one of: a first class where said patient is identified as being at risk for an acute hypotensive episode occurring within a timeframe of a prediction window of w minutes in the future, and a second class where said patient is identified as not being at risk for an acute hypotensive episode, each MAP comprises systolic and diastolic measurements of subjects in both of said first and second classes;
obtaining at least one MAP of an unclassified patient; and
using said classifier system to classify said unclassified patient into one of said first and second classes.

2. The method of claim 1, wherein, for training purposes, using MAPs obtained from systolic and diastolic measurements taken only in a range of 10-60 minutes immediately preceding a start of said prediction window for both first and second classes.

3. The method of claim 1, wherein said training set is from a database containing physiological signals, vitals, and clinical data of subjects in intensive care.

4. The method of claim 1, wherein said MAPs are approximated using systolic (SP) and diastolic (DP) pressures, said approximation comprising:
$$M A P ≅ \frac{2}{3} DP - \frac{1}{3} SP . \qquad$$

5. The method of claim 1, wherein each MAP comprising a time-series signal xₜ, t=1, . . . , N, where N is a total number of systolic and diastolic measurements obtained at time t.

6. The method of claim 5, wherein an acute hypotensive episode is an interval [xᵢ; xᵢ+30] in said time-series signal xₜ in which at least 27 values of said patient's MAP are not greater than 60.

7. The method of claim 1, wherein a first vector consisting of MAPs of subjects in said first class is y₁ and a second vector consisting of MAPs of subjects in said second class is y₂, and wherein μ₁=mean(y₁), μ₂=mean(y₂), σ₁=sd(y₁), σ₂=sd(y₂), k is a value such that μ₁+kσ₁<μ₂−kσ₂, and wherein n₁ is a number of y₁ values above μ₂−kσ₂, n₂ is a number of y₁ values within (μ₁+kσ₁, μ₂−kσ₂), n₃ is a number of y₂ values below μ₁+kσ₁, and n₄ is a number of y₂ values within (μ₁+kσ₁, μ₂−kσ₂).

8. The method of claim 7, wherein, in response to a mean of said unclassified patient's MAPs averaged over at least a one hour time interval immediately preceding a start of said prediction window is less than μ₁+k₀σ₁, where k₀=min(n₁+n₂+n₃+n₄), classifying said unclassified patient into said first class.

9. The method of claim 7, wherein, in response to a mean of said unclassified patient's MAPs averaged over at least a one hour time interval immediately preceding a start of said prediction window is greater than μ₂−k₀σ₂, where k₀=min(n₁+n₂+n₃+n₄), classifying said unclassified patient into said second class.

10. The method of claim 7, wherein, in response to said unclassified patient not being classified into any of said first and second classes, further comprising:
calculating a mean of values of said unclassified patient's MAP averaged over at least a one hour time interval immediately preceding said prediction window; and
in response to said calculated mean falling within (μ₁+kσ₁, μ₂−kσ₂), comparing mean squared deviations of a last of said MAP measurements from all points in said first and second vectors y₁,y₂, said deviations being d₁, d₂, respectively, and in response to d₁>d₂, classifying said unclassified patient to said second class, otherwise classifying said unclassified patient into said first class.

11. The method of claim 1, further comprising adding said classified patient's classification and MAP to said training set.

12. The method of claim 1, further comprising communicating said patient's classification to any of: a display device, a storage device, a wireless handheld device, a laptop, tablet-PC, and a workstation.

13. The method of claim 1, further comprising communicating a notification comprising any of: an audio message, a text message, an email, a phone call, a video, and an alert signal.

14. A system for assessing patient risk for an acute hypotensive episode, the system comprising:
a storage device; and
a processor in communication with said storage device, said processor executing machine readable instructions for implementing a classifier system for classifying an unclassified patient into one of: a first class where said patient is identified as being at risk for an acute hypotensive episode occurring within a timeframe of a prediction window of w minutes in the future, and a second class where said patient is identified as not being at risk for an acute hypotensive episode, said machine readable program instruction for performing:
retrieving, from said storage device, a training set of mean arterial pressures (MAPs) for a plurality of subjects to train a classifier system, each MAP comprises systolic and diastolic measurements of subjects in both of said first and second classes;
retrieving at least one MAP of an unclassified patient; and
classifying said unclassified patient into one of said first and second classes.

15. The system of claim 14, wherein, for training purposes, using MAPs obtained from systolic and diastolic measurements taken only in a range of 10-60 minutes immediately preceding a start of said prediction window for both first and second classes.

16. The system of claim 14, wherein said training set is from a database containing physiological signals, vitals, and clinical data of subjects in intensive care.

17. The system of claim 14, wherein said MAPs are approximated using systolic (SP) and diastolic (DP) pressures, said approximation comprising:
$$M A P ≅ \frac{2}{3} DP - \frac{1}{3} SP . \qquad$$

18. The system of claim 14, wherein each MAP comprising a time-series signal xₜ, t=1, . . . , N, where N is a total number of systolic and diastolic measurements obtained at time t.

19. The system of claim 18, wherein an acute hypotensive episode is an interval [x₁; x₁+30] in said time-series signal xₜ in which at least 27 values of said patient's MAP are not greater than 60.

20. The system of claim 14, wherein a first vector consisting of MAPs of subjects in said first class is y₁ and a second vector consisting of MAPs of subjects in said second class is y₂, and wherein μ₁=mean(y₁), μ₂=mean(y₂), σ₁=sd(y₁), σ₂=sd(y₂), k is a value such that μ₁+kσ₁<μ₂−kσ₂, and wherein n₁ is a number of y₁ values above μ₂−kσ₂, n₂ is a number of y₁ values within (μ₁+kσ₁, μ₂−kσ₂), n₃ is a number of y₂ values below μ₁+kσ₁, and n₄ is a number of y₂ values within (μ₁+kσ₁, μ₂−kσ₂).

21. The system of claim 20, wherein, in response to a mean of said unclassified patient's MAPs averaged over at least a one hour time interval immediately preceding a start of said prediction window is less than μ₁+k₀σ₁, where k₀=min(n₁+n₂+n₃+n₄), classifying said unclassified patient into said first class.

22. The system of claim 20, wherein, in response to a mean of said unclassified patient's MAPs averaged over at least a one hour time interval immediately preceding a start of said prediction window is greater than μ₂ k₀−σ₂, where k₀=min(n₁+n₂+n₃+n₄), classifying said unclassified patient into said second class.

23. The system of claim 20, wherein, in response to said unclassified patient not being classified into any of said first and second classes, further comprising:
calculating a mean of values of said unclassified patient's MAP averaged over at least a one hour time interval immediately preceding said prediction window; and
in response to said calculated mean falling within (μ₁+kσ₁, μ₂−kσ₂), comparing mean squared deviations of a last of said MAP measurements from all points in said first and second vectors y₁,y₂, said deviations being d₁, d₂, respectively, and in response to d₁>d₂, classifying said unclassified patient to said second class, otherwise classifying said unclassified patient into said first class.

24. The system of claim 14, further comprising adding said patient's classification and MAP to said training set.

25. The system of claim 14, further comprising communicating said patient's classification to any of: a display device, a storage device, a wireless handheld device, a laptop, tablet-PC, and a workstation.
